(19) United States
(12) Patent Application Publication
(10) Pub. No.: US 2025/0228432 A1
(43) Pub. Date: Jul. 17, 2025

(54) MEDICAL SYSTEMS, DEVICES, AND RELATED METHODS

(71) Applicant: Boston Scientific Medical Device Limited, Galway (IE)

(72) Inventors: Subodh MOREY, Ponda (IN); Nachiket GOLE, Pune (IN); Samriddhi JAIN, Delhi (IN); Nidhi DHINGRA, Ludhiana (IN); Shrikant Vasant RAUT, Mumbai (IN); Arun ADHIKARATH BALAN, Aluva (IN)

(73) Assignee: Boston Scientific Medical Device Limited, Galway (IE)

(21) Appl. No.: 19/012,153

(22) Filed: Jan. 7, 2025

Related U.S. Application Data
(60) Provisional application No. 63/620,347, filed on Jan. 12, 2024.

(51) Int. Cl. A61B 1/00; A61B 1/012; A61B 1/05; A61B 1/06

(52) U.S. Cl. CPC A61B 1/00096; A61B 1/00174; A61B 1/012; A61B 1/05; A61B 1/0676; A61B 1/0684

(57) ABSTRACT

Medical devices and related methods are described. The medical device may include a handle and a shaft extending distally from the handle. The shaft may have a longitudinal axis and a lumen that terminates at a distal end of the shaft, the distal end including an imaging device. A distal end portion of the shaft may include a transparent or translucent material, and may include a light source, a mirror, and/or a reflector. The light source may be proximal to the distal end of the shaft. The mirror may be longitudinally between the light source and the reflector. The reflector may be proximal to the imaging device.

### CROSS-REFERENCE TO RELATED APPLICATIONS

This application claims the benefit of priority to U.S. Provisional Application No. 63/620,347, filed on Jan. 12, 2024, which is incorporated by reference herein in its entirety.

### TECHNICAL FIELD

Various aspects of the present disclosure relate generally to systems and devices useful in medical procedures. More specifically, the present disclosure relates to systems and devices for illuminating tissue in a medical procedure.

### BACKGROUND

Ureteroscopes, endoscopes, and other medical devices often need to navigate tortuous anatomy. For devices that include a light source, an imaging device, and a working channel opening at the distal tip, implementations of the imaging device and the light source constrain the size of the working channel opening. The size of the working channel limits the types of instruments that may be passed through the working channel.

### SUMMARY

Examples of the present disclosure relate to, among other things, systems and devices for illuminating tissue in a medical procedure. Each of the examples disclosed herein may include one or more of the features described in connection with the disclosed examples.

For example, the present disclosure includes a medical device comprising a handle and a shaft extending distally from the handle and defining a longitudinal axis. The shaft may define a lumen that terminates at a distal end of the shaft, e.g., the distal end including an imaging device. A distal end portion of the shaft may comprise a transparent or translucent material and may include a light source, a mirror, and a reflector. The mirror may be longitudinally between the light source and the reflector. The light source may be proximal to the imaging device.

Any of the medical devices disclosed herein may include any of the following features. The light source may comprise a light emitting diode (LED). The light source may have an annular shape and/or the light source may surround the lumen. The light source may be an RGB LED configured to emit light at different wavelengths. The transparent or translucent material may comprise glass or a polymer. The mirror may form an angle ranging from about 30° to about 60° relative to the longitudinal axis, such as an angle of about 45°. The mirror and/or reflector may comprise an etched portion of the transparent or translucent material or may comprise a metal or metallic coating. For example, the reflector may comprise a metallic coating such as, e.g., a metallic coating comprising silver. In some aspects, the reflector and/or the mirror may have an annular shape. Optionally, the reflector, the light source, and/or the mirror may at least partially or completely surround the lumen. The reflector may be proximal to the imaging device. The mirror and the reflector may be configured to reflect light emitted by the light source through the transparent or translucent material. In at least one example, the shaft has an outermost diameter of about 3.17 mm and/or the lumen has a diameter ranging from about 1 mm to about 2 mm.

The present disclosure also includes a medical device comprising a handle and a shaft extending distally from the handle and defining a longitudinal axis, wherein the shaft defines a lumen that terminates at a distal end of the shaft, the distal end including an imaging device, wherein a distal end portion of the shaft comprises a transparent or translucent material and includes a light source, a mirror, and a reflector, wherein the light source may have an annular shape and may be proximal to the mirror, the reflector, and the imaging device. The light source may comprise an RGB light emitting diode (LED) configured to emit light at different wavelengths. The mirror may comprise an etched portion of the transparent or translucent material and/or the reflector may comprise a metallic coating. The transparent or translucent material may comprise glass or a polymer, for example. The mirror and the reflector may be configured to reflect light emitted by the light source through the transparent or translucent material. At least one or each of the light source, the mirror, and the reflector may at least partially or completely surround the lumen.

The present disclosure also includes a medical device comprising a handle and a shaft extending distally from the handle and defining a longitudinal axis, wherein the shaft defines a lumen that terminates at a distal end of the shaft, the distal end including an imaging device, wherein a distal end portion of the shaft may comprise a transparent or translucent material and include a light source, a mirror, and a reflector. The light source may be proximal to the mirror, the reflector, and the imaging device. The mirror and the reflector may be configured to reflect light emitted by the light source radially outward from the medical device through the transparent or translucent material. At least one or each of the light source, the mirror, and the reflector may surround the lumen. In some aspects, the mirror may be longitudinally between the light source and the reflector.

### BRIEF DESCRIPTION OF THE DRAWINGS

The accompanying drawings, which are incorporated in and constitute a part of this specification, illustrate various exemplary embodiments and together with the description, serve to explain the principles of the disclosure.

FIG. 1 depicts an exemplary medical device according to aspects of this disclosure.

FIG. 2 depicts an exemplary distal end portion of the medical device of FIG. 1, according to aspects of this disclosure.

FIG. 3 depicts components of an exemplary distal end portion of a medical device according to aspects of this disclosure.

FIGS. 4A-4B depict cross-sectional views of the distal end portion of a medical device according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The terms “about,” “approximately,” and like terms (e.g., “substantially”) include values +/−10% of a stated value.

Aspects of the present disclosure provide configurations of the distal end portion of a medical device, including imaging and lighting components with a configuration to increase the relative size of a working channel, e.g., to facilitate treatment of a subject. A medical device, such as an ureteroscope, may be inserted into a body lumen of a subject (e.g., into a kidney) in order to perform a medical procedure (e.g., a laser lithotripsy procedure). The distal end portion may include one or more electronic components, such as light sources (e.g., light emitting diodes (LEDs)), imaging devices (e.g., cameras, other components having imagers, and/or other optical elements such as lenses), and/or other electronic components (e.g., capacitors, diodes, resistors, etc.). Electronic components of the devices herein may include electrical connections, and may also include various elements mounted, for example, or otherwise connected to, the electrical connections. Aspects of the present disclosure may provide for increased illumination at and/or around the distal end portion of a medical device and enlarge the size of a lumen (e.g., working channel) for delivery of instruments to facilitate treatment of a subject.

In some examples herein, including those discussed below, the distal end portion of a medical device (e.g., the distal end portion of a shaft of the medical device) may comprise a transparent or translucent material such that light from a light source within the distal end portion may be directed radially around the shaft. This configuration may increase luminosity within a body lumen and provide for increased size of the lumen (working channel), e.g., due to placement of the light source proximal to the distal end of the shaft.

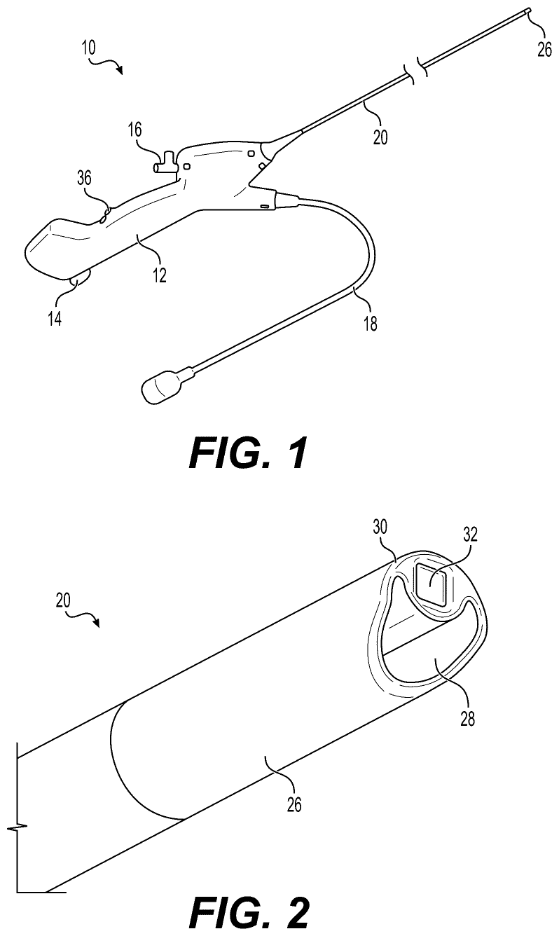

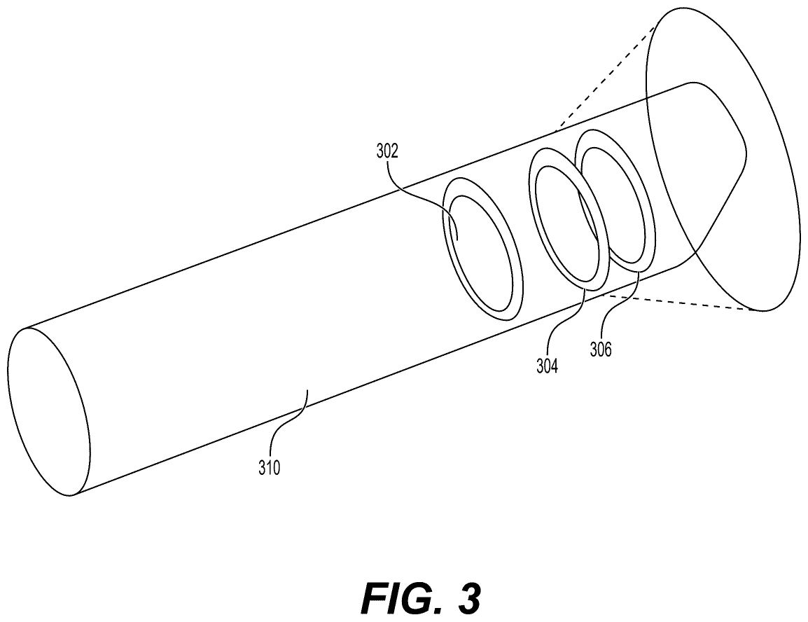

FIGS. 1 and 2 depict aspects of an exemplary medical device 10 according to the present disclosure. Although the discussion herein may refer a ureteroscope as a medical device with the features disclosed herein, it will be appreciated that, unless otherwise specified, the features herein may be used on other suitable medical device such as, e.g., endoscopes, duodenoscopes, gastroscopes, endoscopic ultrasonography (“EUS”) scopes, colonoscopes, bronchoscopes, laparoscopes, cystoscopes, aspiration scopes, and catheters. FIG. 1 depicts a proximal portion of medical device 10, including a handle 12, and a shaft 20 extending distally from the handle 12. FIG. 2 depicts a distal end portion 26 of shaft 20. Handle 12 may be configured for gripping and use by an operator (e.g., a medical professional).

At least a portion of shaft 20 may be flexible, for example, to facilitate navigation through a subject's anatomy, and may define one or more lumens. In some aspects, shaft 20 may include a lumen configured to serve as a working channel for delivery of various instruments and/or material(s) (e.g., fluid) through an opening at a distal end of the shaft 20. Shaft 20 may be configured for at least partial insertion into a body (e.g., a body lumen) of a subject.

As discussed in further detail below, the distal end portion 26 of shaft 20 may include one or more electronic components operably connected to handle 12, e.g., to allow a user control of the electronic component(s) via one or more actuators of the handle 12. Furthermore, one or more articulation members may extend from handle 12 through shaft 20 to articulate shaft 20 in one or more directions. Handle 12 may include an actuator 14 (e.g., lever, switch, button, etc.) coupled to the articulation member(s) to direct shaft 20 in the one or more directions.

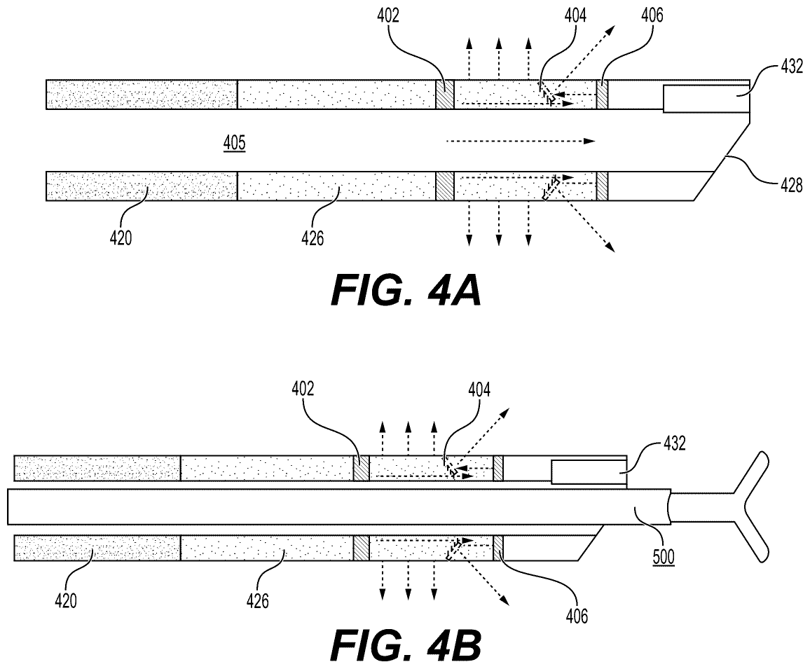

A port 16 of handle 12 may provide access to a lumen (e.g., working channel; see lumen 405 in FIGS. 4A-4B) of shaft 20 of medical device 10. An operator may insert an instrument or deliver a material to the lumen via port 16. As mentioned above, the lumen may extend longitudinally through shaft 20 and terminate at a distal opening 28 of a distal end 30 of shaft 20. Handle 12 may include one or more other actuators, such as actuator 36, operably coupled to an imaging device 32 of shaft 20 to capture still and/or video images. An umbilicus 18 coupled to handle 12 may provide power, signals, etc. to and/or from handle 12. For example, umbilicus 18 may connect medical device 10 to one or more user interfaces, monitors, control units, displays, etc., via handle 12.

As shown in FIG. 2, distal end portion 26 may include a distal end 30 that defines an opening 28 of the lumen (working channel). Instruments and/or other material(s) may be passed through port 16, through the lumen, and extend out of opening 28. An instrument extending distally of opening 28 may be used to perform a medical procedure on the subject (see instrument 500 in FIGS. 4A-4B).

The wall of distal end portion 26 may comprise a transparent or translucent material that allows passage of light therethrough. For example, the transparent or translucent material may comprise glass, a polymer, a polycarbonate, an acrylic, and/or other biocompatible material that permits transmission of light therethrough and radially outward from shaft 20. In some examples, all or a portion of shaft proximal to distal end portion 26 may be opaque.

Distal end 30 of distal end portion 26 may include an imaging device 32 (e.g. a camera, such as a CMOS camera). Imaging device 32 may be configured to take video and/or still images. Imaging device 32 may provide a signal to a monitor or display (e.g., connected via umbilicus 18), so that an operator may view an image provided by imaging device 32 while navigating medical device 10 through a body lumen of a subject. An operator may take photos and/or videos using imaging device 32 via an actuator 36. One of skill in the art will appreciate that imaging device 32 may include various lenses and/or sensors.

FIG. 3 illustrates components of a distal end portion 310 of a shaft of a medical device according to aspects of the present disclosure. Distal end portion 310 may have any or all of the characteristics of distal end portion 26 and/or may be used in combination with any features of medical device 10, except as described below. Distal end portion 310 may include a light source 302, a mirror 304, and a reflector 306. Light source 302 may be proximal to the distalmost end of the shaft, e.g., proximal to an imaging device proximate or at the distal end of the shaft (see, e.g., imaging device 32 in FIG. 2). In some aspects, light source 302 may be proximal to mirror 304 and/or mirror 304 may be proximal to reflector 306. In some aspects, light source 302, mirror 304, and/or reflector 306 may at least partially or complete surround a lumen of the distal end portion 310. For example, light source 302, mirror 304, and/or reflector 306 may be substantially or completely annular in shape.

Distal end portion 310 may comprise a transparent or translucent material, such as glass or a polymer as discussed above in connection to distal end portion 26. Light emitted distally by light source 302 may reflect off of mirror 304 (proximally and/or distally) and may reflect off reflector 306, such that light may pass radially through the transparent or translucent material of distal end portion 310, e.g., through reflection and/or scattering. Mirror 304 and reflector 306 may comprise any suitably reflective material or coating, and may comprise the same or different material(s). Exemplary reflective materials and coatings useful for the present disclosure include, but are not limited to, reflective biocompatible metals such as silver or aluminum. In some aspects, mirror 304 and reflector 306 each may be marmorized surfaces.

In some examples, light source 302 may be an LED, and may feature RGB (red green blue) lighting capabilities such that the light source may emit in a variety of visible wavelengths. For example, light source 302 may initially emit white light and then may transition to emitting a blue light based on user input (e.g., via an actuator of a handle). Tissues may appear differently based on the wavelength of light illuminating the tissue as emitted by light source 302.

While mirror 304 and reflector 306 are depicted as annular components in FIG. 3, in some aspects, mirror 304 and/or reflector 306 may be integrated into the material(s) of distal end portion 310. For example, mirror 304 may comprise an etched portion of the transparent or translucent material that provides an angle relative to a longitudinal axis of the distal end portion 310 in order to reflect light. According to some aspects of the present disclosure, mirror 304 and reflector 306 may be formed from a same portion of transparent or translucent material of distal end portion 310. In some examples, one or both of mirror 304 and/or reflector 306 are separate components coupled to the transparent material(s) of distal end portion 310. For example, mirror 304 and/or reflector 306 may comprise an etched material that is machined/manufactured and assembled into the distal end portion 310. Additionally or alternatively, reflector 306 may comprise a coating on a portion of the transparent or translucent material of distal end portion 310. According to some aspects of the present disclosure, mirror 304 may be configured to reflect light at an angle ranging from about 30° to about 60° relative to the longitudinal axis, such as an angle of about 35°, about 40°, about 45°, about 50°, or about 55°.

According to some aspects of the present disclosure, light source 302, mirror 304, and/or reflector 306 may have an outermost diameter of 9 Fr (3 mm) or less, the width dimension being perpendicular to the longitudinal axis of the shaft and distal end portion 310. For example, distal end portion 310 (and the shaft that includes distal end portion 310) may have an outer diameter of about 9.5 Fr (about 3.17 mm), including a lumen/working channel having a diameter ranging from about 3 Fr to about 6 Fr (about 1-2 mm), e.g., a diameter of about 4.2 Fr (about 1.4 mm). The light source 302, mirror 304, and reflector 306 may be arranged between the lumen/working channel and the outer diameter of distal end portion 310. In some examples, one or more of light source 302, mirror 304, and/or reflector 306 has an annular or ring shape (concentric with the lumen/working channel of distal end portion 310) with an outermost diameter ranging from about 8 Fr to about 9 Fr (about 2.67-3 mm) and an inner diameter ranging from about 3 Fr to about 6 Fr (about 1-2 mm).

FIGS. 4A and 4B illustrate a cross-sectional view of a distal end portion 410 of a shaft according to further aspects of the present disclosure. Distal end portion 410 may have any or all of the characteristics of distal end portion 310 and/or distal end portion 26 as previously described, and may be used in conjunction with any features of medical device 10 as previously described, except as described below. Distal end portion 410 may comprise a transparent or translucent material and define a lumen 405.

Distal end portion 410 includes a light source 402, mirror 404, and reflector 406, which may be annular as shown and surround lumen 405. As discussed above in connection to mirror 304, mirror 404 may be configured to reflect light emitted by light source 402 at an angle to strike reflector 406 and/or pass through the transparent or translucent material of distal end portion 410. Light source 402 may be proximal to mirror 404, such that light emitted by light source 402 distally is reflected. Mirror 404 may be proximal to reflector 406.

FIG. 4B shows an instrument 500 inserted through lumen 405 and extending through distal opening 428 for treatment of a patient. While instrument 500 is illustrated as a forceps, any suitable instrument for treatment may be used.

The total luminosity emitted by a distal end portion of a shaft according to the present disclosure may be substantially increased compared to existing light sources of a scope wherein the light source is located on the distalmost end of the scope.

While principles of this disclosure are described herein with the reference to illustrative examples in a particular context and for particular medical procedures, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A medical device comprising:
a handle; and
a shaft extending distally from the handle and defining a longitudinal axis, wherein the shaft defines a lumen that terminates at a distal end of the shaft, the distal end including an imaging device, wherein a distal end portion of the shaft comprises a transparent or translucent material and includes:
a light source;
a mirror; and
a reflector;
wherein the mirror is longitudinally between the light source and the reflector, the light source being proximal to the imaging device.

2. The medical device of claim 1, wherein the light source comprises a light emitting diode (LED) having an annular shape and surrounding the lumen.

3. The medical device of claim 1, wherein the light source is an RGB LED configured to emit light at different wavelengths.

4. The medical device of claim 1, wherein the transparent or translucent material comprises glass or a polymer.

5. The medical device of claim 1, wherein the mirror forms an angle ranging from about 30° to about 60° relative to the longitudinal axis.

6. The medical device of claim 5, wherein the angle formed by the mirror is about 45°.

7. The medical device of claim 1, wherein the mirror comprises an etched portion of the transparent or translucent material.

8. The medical device of claim 1, wherein the reflector comprises a metallic coating.

9. The medical device of claim 8, wherein the metallic coating comprises silver.

10. The medical device of claim 1, wherein the reflector has an annular shape.

11. The medical device of claim 1, wherein the reflector is proximal to the imaging device.

12. A medical device comprising:
a handle; and
a shaft extending distally from the handle and defining a longitudinal axis, wherein the shaft defines a lumen that terminates at a distal end of the shaft, the distal end including an imaging device, wherein a distal end portion of the shaft comprises a transparent or translucent material and includes:
a light source;
a mirror; and
a reflector;
wherein the light source has an annular shape and is proximal to the mirror, the reflector, and the imaging device.

13. The medical device of claim 12, wherein the light source comprises an RGB light emitting diode (LED) configured to emit light at different wavelengths.

14. The medical device of claim 12, wherein the mirror comprises an etched portion of the transparent or translucent material, and the reflector comprises a metallic coating.

15. The medical device of claim 12, wherein the transparent or translucent material comprises glass or a polymer.

16. The medical device of claim 12, wherein the mirror and the reflector are configured to reflect light emitted by the light source through the transparent or translucent material.

17. The medical device of claim 12, wherein each of the light source, the mirror, and the reflector surrounds the lumen.

18. A medical device comprising:
a handle; and
a shaft extending distally from the handle and defining a longitudinal axis, wherein the shaft defines a lumen that terminates at a distal end of the shaft, the distal end including an imaging device, wherein a distal end portion of the shaft comprises a transparent or translucent material and includes:
a light source;
a mirror; and
a reflector;
wherein the light source is proximal to the mirror, the reflector, and the imaging device; and
wherein the mirror and the reflector are configured to reflect light emitted by the light source radially outward from the medical device through the transparent or translucent material.

19. The medical device of claim 18, wherein each of the light source, the mirror, and the reflector surrounds the lumen.

20. The medical device of claim 18, wherein the mirror is longitudinally between the light source and the reflector.
